(19) United States
(12) Patent Application Publication
(10) Pub. No.: US 2007/0191422 A1
(43) Pub. Date: Aug. 16, 2007

(54) Nicotinic acetylcholine receptor ligands

(75) Inventors: Glen Ernst, Wilmington, DE (US); Robert Jacobs, Triangle Park, NC (US); Eifion Phillips, Boothway, PA (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 10/583,581

(22) PCT Filed: Dec. 20, 2004
(86) PCT No.: PCT/SE04/01943

§ 371 Date: Apr. 6, 2007

Related U.S. Application Data
(60) Provisional application No. 60/531,648, filed on Dec. 22, 2003.

(51) Int. Cl. A61K 31/4745; C07D 453/02

(52) U.S. Cl. 514/305; 546/133

(57) ABSTRACT

Compounds of formula (I), wherein D, Ar1, E and Ar2 are as defined in the specification, processes for preparing them, pharmaceutical compositions containing them and their use in therapy, especially in the treatment or prophylaxis of psychotic and intellectual impairment disorders.

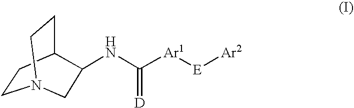

### TECHNICAL FIELD

This invention relates to novel biarylcarboxamides or pharmaceutically-acceptable salts thereof having low P-glycoprotein-mediated efflux, processes for preparing them, pharmaceutical compositions containing them and their use in therapy. This invention particularly relates to compounds having P-glycoprotein-mediated efflux that are ligands for alpha 7 nicotinic acetylcholine receptors (α7 nAChRs).

### BACKGROUND OF THE INVENTION

The use of compounds which bind nicotinic acetylcholine receptors in the treatment of a range of disorders involving reduced cholinergic function, such as Alzheimer's disease, cognitive or attention disorders, anxiety, depression, smoking cessation, neuroprotection, schizophrenia, analgesia, Tourette's syndrome, and Parkinson's disease has been discussed in McDonald et al. (1995) “Nicotinic Acetylcholine Receptors: Molecular Biology, Chemistry and Pharmacology”, Chapter 5 in Annual Reports in Medicinal Chemistry, vol. 30, pp. 41-50, Academic Press Inc., San Diego, Calif.; and in Williams et al. (1994) “Neuronal Nicotinic Acetylcholine Receptors,” Drug News & Perspectives, vol. 7, pp. 205-223.

The facility with which a drug compound gains access to the central nervous system (CNS) substantially impacts whether a compound will have CNS activity. Exclusion of drugs from the CNS is considered to be mediated by the blood-brain barrier (BBB), a single layer of endothelial cells connected by tight junctions. Passive membrane permeability and P-glycoprotein-mediated (PgP) efflux are believed to mechanistically contribute to the BBB and to substantially mediate whether a drug will access or be excluded from the CNS. Thus, high passive membrane permeability and the absence of efflux would likely favor CNS exposure, (Kelly M. Mahar Doan et al., JPET 303 1029-1037, (2002)).

### DESCRIPTION OF THE INVENTION

This invention concerns nicotinic acetylcholine receptor-reactive compounds having surprisingly low P-glycoprotein-mediated efflux in accord with formula I: 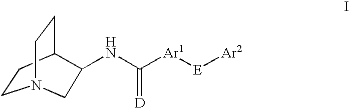
wherein:

D represents oxygen or sulfur;

E represents a single bond, oxygen, sulfur, or NR¹;

Ar₁ is selected from an ortho-substituted 5- or 6-membered aromatic or heteroaromatic ring having 0, 1 or 2 nitrogen atoms, 0 or 1 oxygen atoms, and 0 or 1 sulfur atoms, or selected from an ortho-substituted 8-, 9- or 10-membered fused aromatic or heteroaromatic ring system having 0, 1, 2 or 3 nitrogen atoms, 0 or 1 oxygen atoms, and 0 or 1 sulfur atoms said aromatic or heteroaromatic rings or ring systems having ortho-substituents selected from —C₁-C₆alkyl, —C₂-C₆alkenyl, —C₂-C₆alkynyl, halogen, —CN, —NO₂, —CF₃, —S(O)ₙR², —NR²R³, —CH₂NR²R₃, —OR², —CH₂OR² or —CO₂R⁴;

Ar² is selected from a 5- or 6-membered aromatic or heteroaromatic ring having 0, 1 or 2 nitrogen atoms, 0 or 1 oxygen atoms, and 0 or 1 sulfur atoms;

where Ar² is unsubstituted or has 1, 2 or 3 substituents independently selected from —R², —C₁-C₆alkyl, —C₂-C₆alkenyl, —C₂-C₆alkynyl, halogen, —CN, —NO₂, —CF₃, —S(O)ₙR², —NR²R³, —CH₂NR²R³, —OR², —CH₂OR² or —CO₂R⁴;

R² and R³ are independently selected at each occurrence from hydrogen, —C₁₋C₄alkyl, aryl, heteroaryl, —C(O)R⁴, —C(O)NHR⁴, —CO₂R⁴ or —SO₂R⁴, or

R² and R³ in combination is —(CH₂)ⱼG(CH₂)ₖ—wherein G is oxygen, sulfur, NR4, or a bond;

j is 2, 3 or 4;

k is 0, 1 or 2;

n is 0, 1 or 2, and

R⁴ is independently selected at each occurrence from hydrogen, —C₁-C₄alkyl, aryl, or heteroaryl.

The invention also encompasses stereoisomers, enantiomers, in vivo-hydrolysable precursors and pharmaceutically-acceptable salts of compounds of formula I, pharmaceutical compositions and formulations containing them, methods of using them to treat diseases and conditions either alone or in combination with other therapeutically-active compounds or substances, processes and intermediates used to prepare them, uses of them as medicaments, uses of them in the manufacture of medicaments and uses of them for diagnostic and analytic purposes.

Compound having low P-glycoprotein-mediated efflux of the invention are those according to formula I: 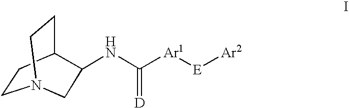
wherein:

D represents oxygen or sulfur;

E represents a single bond, oxygen, sulfur, or NR¹;

Ar¹ is selected from an ortho-substituted 5- or 6-membered aromatic or heteroaromatic ring having 0, 1 or 2 nitrogen atoms, 0 or 1 oxygen atoms, and 0 or 1 sulfur atoms, or selected from an ortho-substituted 8-, 9- or 10-membered fused aromatic or heteroaromatic ring system having 0, 1, 2 or 3 nitrogen atoms, 0 or 1 oxygen atoms, and 0 or 1 sulfur atoms, said aromatic or heteroaromatic rings or ring systems having ortho-substituents selected from —C₁-C₆alkyl, —C₂-C₆alkenyl, —C₂-C₆alkynyl, halogen, —CN, —NO₂, —CF₃, —S(O)ₙR², —NR²R³, —CH₂NR²R³, —OR², —CH₂R² or —CO₂R⁴;

Ar² is selected from a 5- or 6-membered aromatic or heteroaromatic ring having 0, 1 or 2 nitrogen atoms, 0 or 1 oxygen atoms, and 0 or 1 sulfur atoms;

where Ar² is unsubstituted or has 1, 2 or 3 substituents independently selected from —R², —C₁-C₆alkyl, —C₂-C₆alkenyl, —C₂-C₆alkynyl, halogen, —CN, —NO₂, —CF₃, —S(O)ₙR², —NR²R³, —CH₂NR²R³, —OR², —CH₂OR² or —CO₂R⁴;

R² and R³ are independently selected at each occurrence from hydrogen, —C₁-C₄alkyl, aryl, heteroaryl, —C(O)R⁴, —C(O)NHR⁴, —CO₂R⁴ or —SO₂R⁴, or

R² and R³ in combination is —(CH₂)ⱼG(CH₂)ₖ—wherein G is oxygen, sulfur, NR⁴, or a bond;

j is 2, 3 or 4;

k is 0, 1 or 2;

n is 0, 1 or 2, and

R⁴ is independently selected at each occurrence from hydrogen, —C₁-C₄alkyl, aryl, or heteroaryl, and

stereoisomers, enantiomers, in vivo-hydrolysable precursors and pharmaceutically-acceptable salts thereof.

Particular compounds of the invention are R-isomers of compounds of formula I in accord with formula II, 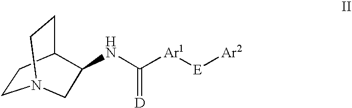
wherein D, Ar¹, E and Ar² are as defined for compounds of formula I.

Other particular compounds of the invention are those according to formula I wherein:

D represents oxygen or sulfur;

E represents a single bond, oxygen, sulfur, or NR¹;

Ar¹ is selected from an ortho-substituted 5- or 6-membered aromatic or heteroaromatic ring having 0, 1 or 2 nitrogen atoms, 0 or 1 oxygen atoms, and 0 or 1 sulfur atoms, or selected from an ortho-substituted 8-, 9- or 10-membered fused aromatic or heteroaromatic ring system having 0, 1, 2 or 3 nitrogen atoms, 0 or 1 oxygen atoms, and 0 or 1 sulfur atoms, said aromatic or heteroaromatic rings or ring systems having ortho-substituents selected from —C₁-C₆alkyl, halogen, —CN, —NO₂, —CF₃, —NR²R³, —OR², or —CO₂R⁴;

Ar² is selected from a 5- or 6-membered aromatic or heteroaromatic ring having 0, 1 or 2 nitrogen atoms, 0 or 1 oxygen atoms, and 0 or 1 sulfur atoms;

where Ar² is unsubstituted or has 1, 2 or 3 substituents independently selected from —R², —C₁-C₆alkyl, —C₂-C₆alkenyl, —C₂-C₆alkynyl, halogen, —CN, —NO₂, —CF₃, —S(O)ₙR², —NR²R³, —CH₂NR²R³, —OR², —CH₂OR² or —CO₂R4;

R² and R³ are independently selected at each occurrence from hydrogen, —C₁-C₄alkyl, aryl, heteroaryl, —C(O)R⁴, —C(O)NHR⁴, —CO₂R⁴ or —SO₂R⁴, or

R² and R³ in combination is —(CH₂)ⱼG(CH₂)ₖ—wherein G is oxygen, sulfur, NR⁴, or a bond;

j is 2, 3 or 4;

k is 0, 1 or 2;

n is 0, 1 or 2, and

R⁴ is independently selected at each occurrence from hydrogen, —C₁-C₄alkyl, aryl, or heteroaryl, and

stereoisomers, enantiomers, in vivo-hydrolysable precursors and pharmaceutically-acceptable salts thereof.

More particular compounds of the invention are those according to formula I wherein:

D represents oxygen;

E represents a single bond;

Ar¹ is selected from an ortho-substituted 5- or 6-membered aromatic or heteroaromatic ring having 0, 1 or 2 nitrogen atoms, 0 or 1 oxygen atoms, and 0 or 1 sulfur atom, said aromatic or heteroaromatic rings or having ortho-substituents selected from —C₁-C₆alkyl, halogen, —CN, —NO₂, —CF₃, —NR²R³, —OR² or —CO₂R⁴;

Ar² is selected from a 5- or 6-membered aromatic or heteroaromatic ring having 0, 1 or 2 nitrogen atoms, 0 or 1 oxygen atoms, and 0 or 1 sulfur atoms, and

stereoisomers, enantiomers, in vivo-hydrolysable precursors and pharmaceutically-acceptable salts thereof.

Even more particular compounds of the invention are those according to formula I wherein:

D represents oxygen;

E represents a single bond;

Ar¹ is selected from an ortho-substituted 5- or 6-membered aromatic or heteroaromatic ring having 0, 1 or 2 nitrogen atoms, 0 or 1 oxygen atoms, and 0 or 1 sulfur atom, said aromatic or heteroaromatic ring having ortho-substituents selected from —CN, —NO₂, —CF₃, or —OR²;

Ar² is selected from phenyl or pyridyl, and

stereoisomers, enantiomers, in vivo-hydrolysable precursors and pharmaceutically-acceptable salts thereof.

Other particular compounds of the invention include those of formula I wherein D is O; or an enantiomer thereof, and pharmaceutically-acceptable salts thereof.

Other particular compounds of the invention include those of formula I wherein Ar¹ is selected from phenyl or thiophenyl and Ar² is selected from phenyl, pyridyl, furanyl or thiophenyl having optional substituents as defined herein.

Particular compounds of the invention are those described herein and pharmaceutically-acceptable salts thereof.

In a further aspect the invention relates to compounds according to formula I wherein one or more of the atoms is a radioisotope of the same element. In a particular form of this aspect of the invention the compound of formula I is labeled with tritium. Such radio-labeled compounds are synthesized either by incorporating radio-labeled starting materials or, in the case of tritium, exchange of hydrogen for tritium by known methods. Known methods include (1) electrophilic halogenation, followed by reduction of the halogen in the presence of a tritium source, for example, by hydrogenation with tritium gas in the presence of a palladium catalyst, or (2) exchange of hydrogen for tritium performed in the presence of tritium gas and a suitable organometallic (e.g. palladium) catalyst.

Compounds of the invention labeled with tritium are useful for the discovery of novel medicinal compounds which bind to and modulate the activity, by agonism, partial agonism, or antagonism, of the α7 nicotinic acetylcholine receptor. Such tritium-labeled compounds may be used in assays that measure the displacement of a such compounds to assess the binding of ligands that bind to α7 nicotinic acetylcholine receptors.

In another aspect the invention relates to compounds according to formula I and their use in therapy and to compositions containing them.

In another aspect the invention encompasses the use of compounds according to formula I for the therapy of diseases mediated through the action of nicotinic acetylcholine receptors. A more particular aspect of the invention relates to the use of compounds of formula I for the therapy of diseases mediated through the action of α7 nicotinic acetylcholine receptors.

Another aspect of the invention encompasses a method of treatment or prophylaxis of diseases or conditions in which activation of the α7 nicotinic receptor is beneficial which method comprises administering a therapeutically-effective amount of a compound of the invention to a subject suffering from said disease or condition.

One embodiment of this aspect of the invention is a method of treatment or prophylaxis, wherein the disorder is anxiety, schizophrenia, mania or manic depression.

Another embodiment of this aspect of the invention is a method of treatment or prophylaxis of neurological disorders, psychotic disorders or intellectual impairment disorders, which comprises administering a therapeutically effective amount of a compound of the invention.

Another embodiment of this aspect of the invention is a method of treatment or prophylaxis, wherein the disorder is Alzheimer's disease, learning deficit, cognition deficit, attention deficit, memory loss, or Attention Deficit Hyperactivity Disorder.

Another embodiment of this aspect of the invention is a method of treatment or prophylaxis, wherein the disorder is Parkinson's disease, Huntington's disease, Tourette's syndrome, or neurodegenerative disorders in which there is loss of cholinergic synapses.

Another embodiment of this aspect of the invention is a method of treatment or prophylaxis of jetlag, nicotine addiction, craving, pain, and for ulcerative colitis, which comprises administering a therapeutically effective amount of a compound of the invention.

Yet another embodiment of this aspect of the invention is a method for inducing the cessation of smoking which comprises administering an effective amount of a compound of the invention.

Another embodiment of this aspect of the invention is a pharmaceutical composition comprising a compound of the invention and a pharmaceutically-acceptable diluent, lubricant or carrier.

A further aspect of the invention relates to a pharmaceutical composition useful for treating or preventing a condition or disorder mentioned herein arising from dysfunction of nicotinic acetylcholine receptor neurotransmission in a mammal, preferably a human, comprising an amount of a compound of formula I, an enantiomer thereof or a pharmaceutically-acceptable salt thereof, effective in treating or preventing such disorder or condition, and pharmaceutically-acceptable additives carrier.

Another embodiment of this aspect of the invention relates to use of a pharmaceutical composition of the invention for the treatment, amelioration or prophylaxis of human diseases or conditions in which activation of the α7 nicotinic receptor is beneficial.

Another embodiment of this aspect of the invention is the use of the pharmaceutical composition of the invention for the treatment or prophylaxis of neurological disorders, psychotic disorders or intellectual impairment disorders.

Another embodiment of this aspect of the invention is the use of the pharmaceutical composition of the invention for the treatment or prophylaxis of Alzheimer's disease, learning deficit, cognition deficit, attention deficit, memory loss, Attention Deficit Hyperactivity Disorder, anxiety, schizophrenia, or mania or manic depression, Parkinson's disease, Huntington's disease, Tourette's syndrome, neurodegenerative disorders in which there is loss of cholinergic synapse, jetlag, cessation of smoking, nicotine addiction including that resulting from exposure to products containing nicotine, craving, pain, and for ulcerative colitis.

A further aspect of the invention is the use of a compound according to the invention, an enantiomer thereof or a pharmaceutically-acceptable salt thereof, in the manufacture of a medicament for the treatment or prophylaxis of the diseases or conditions mentioned herein.

Another embodiment of this aspect of the invention is the use of a compound of the invention in the manufacture of a medicament for the treatment or prophylaxis of human diseases or conditions in which activation of the α7 nicotinic receptor is beneficial.

Another embodiment of this aspect of the invention is the use of a compound of the invention in the manufacture of a medicament for the treatment or prophylaxis of neurological disorders, psychotic disorders or intellectual impairment disorders.

Another embodiment of this aspect of the invention is the use of a compound of the invention in the manufacture of a medicament for treatment or prophylaxis of Alzheimer's disease, learning deficit, cognition deficit, attention deficit, memory loss or Attention Deficit Hyperactivity Disorder.

Another embodiment of this aspect of the invention is the use of a compound of the invention in the manufacture of a medicament for treatment or prophylaxis of anxiety, schizophrenia, or mania or manic depression.

Another embodiment of this aspect of the invention is the use of a compound of the invention in the manufacture of a medicament for treatment or prophylaxis of Parkinson's disease, Huntington's disease, Tourette's syndrome, or neurodegenerative disorders in which there is loss of cholinergic synapses.

Another embodiment of this aspect of the invention is the use of a compound as described above in the manufacture of a medicament for the treatment or prophylaxis of jetlag, pain, or ulcerative colitis.

Another aspect of the invention relates to the use of a compound of the invention in the manufacture of a medicament for facilitating the cessation of smoking or the treatment of nicotine addiction or craving including that resulting from exposure to products containing nicotine.

For the uses, methods, medicaments and compositions mentioned herein the amount of compound used and the dosage administered will, of course, vary with the compound employed, the mode of administration and the treatment desired. However, in general, satisfactory results are obtained when the compounds of the invention are administered at a daily dosage of from about 0.1 mg to about 20 mg/kg of animal body weight. Such doses may be given in divided doses 1 to 4 times a day or in sustained release form. For man, the total daily dose is in the range of from 5 mg to 1,400 mg, more preferably from 10 mg to 100 mg, and unit dosage forms suitable for oral administration comprise from 2 mg to 1,400 mg of the compound admixed with a solid or liquid pharmaceutical carriers, lubricants and diluents.

The compounds of formula I, an enantiomer thereof, and pharmaceutically-acceptable salts thereof, may be used on their own or in the form of appropriate medicinal preparations for enteral or parenteral administration. According to a further aspect of the invention, there is provided a pharmaceutical composition including preferably less than 80% and more preferably less than 50% by weight of a compound of the invention in admixture with an inert pharmaceutically-acceptable diluent, lubricant or carrier. Examples of diluents, lubricants and carriers are:
for tablets and dragees: lactose, starch, talc, stearic acid;
for capsules: tartaric acid or lactose;
for injectable solutions: water, alcohols, glycerin, vegetable oils;
for suppositories: natural or hardened oils or waxes.

There is also provided a process for the preparation of such a pharmaceutical composition which process comprises mixing the ingredients.

Compounds according to the invention are agonists of nicotinic acetylcholine receptors. While not being limited by theory, it is believed that agonists of the α7 nicotinic acetylcholine receptor (nAChR) subtype are useful in the treatment or prophylaxis of neurological disorders, psychotic disorders and intellectual impairment disorders, and to have advantages over compounds which are or are also agonists of the α4 nAChR subtype. Therefore, compounds which are selective for the α7 nAChR subtype are preferred. The compounds of the invention are indicated as pharmaceuticals, in particular in the treatment or prophylaxis of neurological disorders, psychotic disorders and intellectual impairment disorders. Examples of psychotic disorders include schizophrenia, mania and manic depression, and anxiety. Examples of intellectual impairment disorders include Alzheimer's disease, learning deficit, cognition deficit, attention deficit, memory loss, and Attention Deficit Hyperactivity Disorder. The compounds of the invention may also be useful as analgesics in the treatment of pain, chronic pain, and in the treatment or prophylaxis of Parkinson's disease, Huntington's disease, Tourette's syndrome, and neurodegenerative disorders in which there is loss of cholinergic synapses.

Compounds of the invention may further useful for the treatment or prophylaxis of jetlag, for use in inducing the cessation of smoking, craving, and for the treatment or prophylaxis of nicotine addiction including that resulting from exposure to products containing nicotine.

It is also believed that compounds according to the invention are useful in the treatment and prophylaxis of ulcerative colitis.

The compounds of the invention have the advantage that they may be less toxic, be more efficacious, be longer acting, have a broader range of activity, be more potent, produce fewer side effects, are more easily absorbed or have other useful pharmacological properties.

The compounds of formula I exist in tautomeric or enantiomeric forms, all of which are included within the scope of the invention. The various optical isomers may be isolated by separation of a racemic mixture of the compounds using conventional techniques, e.g. fractional crystallization, or chiral HPLC. Alternatively the individual enantiomers may be made by reaction of the appropriate optically active starting materials under reaction conditions which will not cause racemization.

General Experimental Procedures and Definitions

Commercial reagents were used without further purification. Mass spectra were recorded using either a Hewlett Packard 5988A or a MicroMass Quattro-1 Mass Spectrometer and are reported as m/z for the parent molecular ion. Room temperature refers to 20-25° C.

SiO₂ chromatography was performed with an Isco CombiFlash Sq 16× instrument and pre-packaged disposable RediSep SiO₂ stationary phase columns (4, 12, 40, 120 gram sizes) with gradient elution at 5-125 mL/min of selected bi-solvent mixture, UV detection (190-760 nm range) or timed collection, 0.1 mm flow cell path length.

Microwave heating was achieved with a Personal Chemistry Smith Synthesizer or a Personal Chemistry Emrys Optimizer (monomodal, 2.45 GHz, 300 W max).

Supercritical Fluid Chromatography (SFC) was performed as a means of purification for selected compounds and intermediates.

Reverse Phase High Pressure Liquid Chromatography (RP-HPLC) was employed as a method of purification for selected compounds.

LC/MS HPLC method was generally performed with a Agilent Zorbax 5μ SB-C8 column 2.1 mm×5 cm. Solvents: A=H₂O with 0.05% TFA, B=10% H₂O, 90% Acetonitrile, 0.05% TFA. Gradient: (10-90% B over 3 min., 90% B hold through 4 min., −10% B at 5 min. and hold at 10% B until 6 min).

Unless otherwise indicated, halo includes chloro, bromo, fluoro and iodo; C₁₋₆alkyl includes methyl, ethyl and linear, cyclic or branched propyl, butyl, pentyl or hexyl; C₂₋₆alkenyl includes ethenyl, 1-propenyl, 2-propenyl or 3-propenyl and linear, branched or cyclic butenyl, pentenyl or hexenyl; C₂₋₆alkynyl includes ethynyl or propynyl; the C₁₋₄alkyl groups referred to herein, e.g., methyl, ethyl, n-propyl, n-butyl, i-propyl, i-butyl, t-butyl, s-butyl, whether alone or part of another group, may be straight-chained or branched, and the C₃₋₄ alkyl groups may also be cyclic, e.g., cyclopropyl, cyclobutyl. Alkyl groups referred to herein may optionally have one, two or three halogen atoms substituted thereon.

Unless otherwise indicated, aryl refers to a phenyl ring which may optionally be substituted with one to three of the following substituents selected from: halogen, C₁₋₄alkyl, C₂₋₄alkenyl, C₂₋₄alkynyl, NR¹R², CH₂NR¹R², OR³, CH₂OR³, CO₂R⁴, CN, NO₂, and CF₃.

Unless otherwise indicated, heteroaryl refers to a 5- or 6-membered aromatic or heteroaromatic ring containing zero to three nitrogen atoms, zero or one oxygen atom, and zero or one sulfur atom, provided that the ring contains at least one nitrogen, oxygen, or sulfur atom, which may optionally be substituted with one or more substituents selected from: halogen, C₁₋₄alkyl, C₂₋₄alkenyl, C₂₋₄alkynyl, NR¹R², CH₂NR¹R², OR³, CH₂OR³, CO₂R⁴, CN, NO₂, and CF₃.

Unless otherwise indicated, halogen refers to fluorine, chlorine, bromine, or iodine.

Pharmaceutically-acceptable derivatives include solvates and salts. For example, the compounds of formula I can form acid addition salts with acids, such as the conventional pharmaceutically-acceptable acids, for example, maleic, hydrochloric, hydrobromic, phosphoric, acetic, fumaric, salicylic, citric, lactic, mandelic, tartaric and methanesulfonic acids.

Pharmacology

The pharmacological activity of the compounds of the invention may be measured in the tests set out below:

Test A—Assay for affinity at α₇ nAChR subtype

¹²⁵I-α—Bungarotoxin (BTX) binding to rat hippocampal membranes.

Rat hippocampi are homogenized in 20 volumes of cold homogenisation buffer (HB: concentrations of constituents (mM): tris(hydroxymethyl)aminomethane 50; MgCl₂ 1; NaCl 120; KCl 5: pH 7.4). The homogenate is centrifuged for 5 minutes at 1000×g, the supernatant saved and the pellet re-extracted. The pooled supernatants are centrifuged for 20 minutes at 12000×g, washed, and re-suspended in HB. Membranes (30-80 μg) are incubated with 5 nM [¹²⁵I]α-BTX, 1 mg/mL BSA (bovine serum albumin), test drug, and either 2 mM CaCl₂ or 0.5 mM EGTA [ethylene glycol-bis(β-aminoethylether)] for 2 hours at 21° C., and then filtered and washed 4 times over Whatman glass fiber filters (thickness C) using a Brandel cell harvester. Pre-treating the filters for 3 hours with 1% (BSA/0.01% PEI (polyethyleneimine) in water is critical for low filter blanks (0.07% of total counts per minute). Non-specific binding is described by 100 μM (−)-nicotine, and specific binding is typically 75%.

Test B—Assay for affinity to the α₄ nAChR subtype

[³H]-(−)-nicotine binding.

Using a procedure modified from Martino-Barrows and Kellar (Mol Pharm (1987) 31:169-174), rat brain (cortex and hippocampus) is homogenised as in the [¹²⁵I]α-BTX binding assay, centrifuged for 20 minutes at 12,000×g, washed twice, and then re-suspended in HB containing 100 μM diisopropyl fluorophosphate. After 20 minutes at 4° C., membranes (approximately 0.5 mg) are incubated with 3 nM [³H]-(−)-nicotine, test drug, 1 μM atropine, and either 2 mM CaCl2 or 0.5 mM EGTA for 1 hour at 4° C., and then filtered over Whatman glass fiber filters (thickness C) (pre-treated for 1 hour with 0.5% PEI) using a Brandel cell harvester. Non-specific binding is described by 100 μM carbachol, and specific binding is typically 84%.

Binding data analysis for Tests A and B

IC50 values and pseudo Hill coefficients (n_{H}) are calculated using the non-linear curve fitting program ALLFIT (DeLean A, Munson P J and Rodbard D (1977) Am. J. Physiol., 235:E97-E102). Saturation curves are fitted to a one site model, using the non-linear regression program ENZFITTER (Leatherbarrow, R. J. (1987)), yielding K_{D} values of 1.67 and 1.70 nM for the ¹²⁵I-α-BTX and [³H]-(−)-nicotine ligands respectively. Kᵢ values are estimated using the general Cheng-Prusoff equation:
Kᵢ=IC₅₀/((2+([ligand]/K_{D})ⁿ)^{1/n}−1)
where a value of n=1 is used whenever n_{H}<1.5 and a value of n=2 is used when n_{H}>1.5. Samples are assayed in triplicate and were typically ±5%. Kᵢ values are determined using 6 or more drug concentrations. The compounds of the invention are compounds with binding affinities (Kᵢ) of less than 10 μM in either Test A or Test B, indicating that they are expected to have useful therapeutic activity.
Test C—Assay for P-glycoprotein-mediated efflux

P-glycoprotein-mediated (Pgp) transport is assayed in Madin-Darby Canine Kidney Cells Expressing Human P-glycoprotein (MDR1-MDCK) cells as follows.

MDR1-MDCK cell lines are maintained in culture in Dulbecco's Minimal Essential Medium (DMEM) containing 10% Fetal Bovine Serum (FBS) at 37° C. and 5% CO₂ and are passaged twice weekly.

To perform the assay, cells are seeded into the apical side (A) of 12-well Costar plates at 0.5 mL per well at a cell density of 300,000 cells per mL or into 24-well Falcon plates at 0.4 mL per well at a cell density of 150,000 cells per mL and 1.5 mL (12-well plates) or 1 mL (24-well plates) of medium is added to the transwell basolateral (B) chambers. The medium is replaced daily and monolayers are used for transport assays 3 days post seeding. Monolayers are fed 2 h prior to performing a transport assay.

Chopstick electrodes are positioned to contact the medium on both sides of a monolayer and the resistance across the monolayer is determined. Normal values for the resistance across a monolayer are 130 to 160 Ohms/cm².

Transport assays are performed manually with 12-well plates and run in basolateral to apical (B to A) and apical to basolateral (A to B) directions in triplicate. Test compounds are dissolved in DMSO and diluted to the test concentrations with HBSS with the final concentration of DMSO in test solutions <1%. Transwells are washed with HBSS at 37° C. for 20 to 40 min and complement plates are prepared.

For A to B experiments, 1.5 mL of HBSS is added to the well followed by 0.5 mL test solution to the insert. For B to A experiments, 1.5 mL test solution is added to the well followed by 0.5 mL HBSS to the insert. The inserts are transferred to the complement plate and the plates incubated in a 37° C. water bath with a shaking rate of 70 rpm for 60 min. At the end of each experiment, the inserts are removed from the plates and samples transferred from both donor and receiver chambers to HPLC vials and analyzed by conventional LC/MS/MS methods. Calibration standards of 0, 0.005, 0.05, and 0.5 μM are used. Calculation of Results:

The apparent permeability is calculated according to the following equations:
Papp=[(Vr×Cr)÷(A×t×Co)]×1,000,000 (10⁻⁶ cm/sec)
Flux Ratio=Papp_{(B to A)}÷PAPP_{(A to B)}
MB(% Recovery)={[(Vr×Cr)+(Vd×Cd)]÷(Vd×Co)}×100
Where: Vr=Volume of receiver cm³; Cr=Concentration in receiver at 60 min; Co=Initial concentration in donor; Vd=Volume of donor; Cd=Concentration in donor at 60 min; A=Surface area of Transwells and t=60 min.

Compounds of the invention generally have an A-B/B-A ratio of less than 2.5 in this test.

Compounds of the Invention

Compounds of the invention may be prepared by reacting suitable substituted aromatic or heteroaromatic carboxylic acids (0.50 mmol), R-(+)-3-aminoquinuclidine dihydrochloride (100 mg, 0.50 mmol), 1-hydroxybenzotriazole hydrate (68 mg, 0.50 mmol), O-(benzotriazol-1-yl)-N,N,N′,N′-tetramethyluronium tetrafluoroborate (161 mg, 0.50 mmol) and diisopropylethylamine (0.35 mL, 2.0 mmol) in dry N,N-dimethylformamide (2 mL) at ambient temperature for 23 h. Reaction mixtures are poured into 1 N sodium hydroxide solution and extracted with ethyl acetate (3×). Ethyl acetate layers are combined and washed with 1 N NaOH (1×), water (4×), brine (1×), and dried over MgSO₄. After filtration, the solvent is removed in vacuo to yield the desired compound.

The following examples are non-limiting and embody particular aspects of the invention.

N-(R)-1-Azabicyclo[2.2.2]oct-3-yl-2-methyl-5-phenylbenzamide;

N-(R)-1-Azabicyclo[2.2.2]oct-3-yl-2-methyl-3-phenylbenzamide;

(N-(R)-1-Azabicyclo[2.2.2]oct-3-yl)-3-methyl-5-phenylthiophene-2-carboxylic acid amide;

(N-(R)-1-Azabicyclo[2.2.2]oct-3-yl)-3-methyl-5-(3-pyridyl)thiophene-2-carboxylic acid amide;

N-(R)-1-Azabicyclo[2.2.2]oct-3-yl-2-carboxy-5-phenylbenzamide;

N-(R)-1-Azabicyclo[2.2.2]oct-3-yl-2-carboxy-3-phenylbenzamide;

(N-(R)-1-Azabicyclo[2.2.2]oct-3-yl)-3-carboxy-5-phenylthiophene-2-carboxylic acid amide;

(N-(R)-1-Azabicyclo[2.2.2]oct-3-yl)-3-carboxy-5-(3-pyridyl)thiophene-2-carboxylic acid amide;

N-(R)-1-Azabicyclo[2.2.2]oct-3-yl-2-cyano-5-phenylbenzamide;

N-(R)-1-Azabicyclo[2.2.2]oct-3-yl-2-cyano-3-phenylbenzamide;

(N-(R)-1-Azabicyclo[2.2.2]oct-3-yl)-3-cyano-5-phenylthiophene-2-carboxylic acid amide;

(N-(R)-1-Azabicyclo[2.2.2]oct-3-yl)-3-cyano-5-(3-pyridyl)thiophene-2-carboxylic acid amide;

N-(R)-1-Azabicyclo[2.2.2]oct-3-yl-2-amino-5-phenylbenzamide;

N-(R)-1-Azabicyclo[2.2.2]oct-3-yl-2-amino-3-phenylbenzamide;

(N-(R)-1-Azabicyclo[2.2.2]oct-3-yl)-3-amino-5-phenylthiophene-2-carboxylic acid amide, and

(N-(R)-1-Azabicyclo [2.2.2]oct-3-yl)-3-amino-5-(3-pyridyl)thiophene-2-carboxylic acid amide.

1. A compound having low P-glycoprotein-mediated efflux according to formula I: 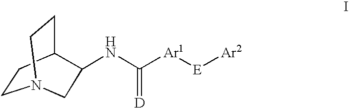
wherein:
D represents oxygen or sulfur;
E represents a single bond, oxygen, sulfur, or NR¹;
Ar¹ is selected from an ortho-substituted 5- or 6-membered aromatic or heteroaromatic ring having 0, 1 or 2 nitrogen atoms, 0 or 1 oxygen atoms, and 0 or 1 sulfur atoms, or selected from an ortho-substituted 8-, 9- or 10-membered fused aromatic or heteroaromatic ring system having 0, 1, 2 or 3 nitrogen atoms, 0 or 1 oxygen atoms, and 0 or 1 sulfur atoms, said aromatic or heteroaromatic rings or ring systems having ortho-substituents selected from —C₁-C₆alkyl, —C₂-C₆alkenyl, —C₂-C₆alkynyl, halogen, —CN, —NO₂, —CF₃, —S(O)ₙR², —NR²R³, —CH₂NR²R³, —OR², —CH₂OR² or —CO₂R⁴;
Ar² is selected from a 5- or 6-membered aromatic or heteroaromatic ring having 0, 1 or 2 nitrogen atoms, 0 or 1 oxygen atoms, and 0 or 1 sulfur atoms;
where Ar² is unsubstituted or has 1, 2 or 3 substituents independently selected from —R², —C₁-C₆alkyl, —C₂-C₆alkenyl, —C₂-C₆alkynyl, halogen, —CN, —NO₂, —CF₃, —S(O)ₙR², —NR²R³, —CH₂NR²R³, —OR², —CH₂OR² or —CO₂R⁴;
R² and R³ are independently selected at each occurrence from hydrogen, —C₁₋C₄alkyl, aryl, heteroaryl, —C(O)R⁴, —C(O)NHR⁴, —CO₂R⁴ or —SO₂R⁴, or
R² and R³ in combination is —(CH₂)ⱼG(CH₂)ₖ—wherein G is oxygen, sulfur, NR⁴, or a bond;
j is 2, 3 or 4;
k is 0, 1 or 2;
n is 0, 1 or 2, and
R⁴ is independently selected at each occurrence from hydrogen, —C₁-C₄alkyl, aryl, or heteroaryl, and
stereoisomers, enantiomers, in vivo-hydrolysable precursors and pharmaceutically-acceptable salts thereof.

2. A compound according to claim 1 being an R-isomers of a compound of formula I in accord with formula II, 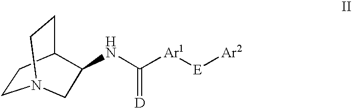
wherein D, Ar¹, E and Ar² are as defined for compounds of formula I.

3. A compound according to claim 1, wherein:
D represents oxygen or sulfur;
E represents a single bond, oxygen, sulfur, or NR¹;
Ar¹ is selected from an ortho-substituted 5- or 6-membered aromatic or heteroaromatic ring having 0, 1 or 2 nitrogen atoms, 0 or 1 oxygen atoms, and 0 or 1 sulfur atoms, or selected from an ortho-substituted 8-, 9- or 10-membered fused aromatic or heteroaromatic ring system having 0, 1, 2 or 3 nitrogen atoms, 0 or 1 oxygen atoms, and 0 or 1 sulfur atoms, said aromatic or heteroaromatic rings or ring systems having ortho-substituents selected from —C₁-C₆alkyl, halogen, —CN, —NO₂, —CF₃, —NR²R³, —OR², or —CO₂R⁴;
Ar² is selected from a 5- or 6-membered aromatic or heteroaromatic ring having 0, 1 or 2 nitrogen atoms, 0 or 1 oxygen atoms, and 0 or 1 sulfur atoms;
where Ar² is unsubstituted or has 1, 2 or 3 substituents independently selected from —R², —C₁-C₆alkyl, —C₂-C₆alkenyl, —C₂-C₆alkynyl, halogen, —CN, —NO₂, —CF₃, —S(O)ₙR², —NR²R³, —CH₂NR²R³, —OR², —CH₂OR² or —CO₂R⁴;
R² and R³ are independently selected at each occurrence from hydrogen, —C₁-C₄alkyl, aryl, heteroaryl, —C(O)R⁴, —C(O)NHR⁴, —CO₂R⁴ or —SO₂R⁴, or
R² and R³ in combination is —(CH₂)ⱼG(CH₂)ₖ—wherein G is oxygen, sulfur, NR⁴, or a bond;
j is 2, 3 or 4;
k is 0, 1 or 2;
n is 0, 1 or 2, and
R⁴ is independently selected at each occurrence from hydrogen, —C₁-C₄alkyl, aryl, or heteroaryl, and
stereoisomers, enantiomers, in vivo-hydrolysable precursors and pharmaceutically-acceptable salts thereof.

4. A compound according to claim 1, wherein:
D represents oxygen;
E represents a single bond;
Ar¹ is selected from an ortho-substituted 5- or 6-membered aromatic or heteroaromatic ring having 0, 1 or 2 nitrogen atoms, 0 or 1 oxygen atoms, and 0 or 1 sulfur atom, said aromatic or heteroaromatic rings or having ortho-substituents selected from —C₁-C₆alkyl, halogen, —CN, —NO₂, —CF₃, —NR²R³, —OR² or —CO₂R⁴;
Ar² is selected from a 5- or 6-membered aromatic or heteroaromatic ring having 0, 1 or 2 nitrogen atoms, 0 or 1 oxygen atoms, and 0 or 1 sulfur atoms, and
stereoisomers, enantiomers, in vivo-hydrolysable precursors and pharmaceutically-acceptable salts thereof.

5. A compound according to claim 1, wherein:
D represents oxygen;
E represents a single bond;
Ar¹ is selected from an ortho-substituted 5- or 6-membered aromatic or heteroaromatic ring having 0, 1 or 2 nitrogen atoms, 0 or 1 oxygen atoms, and 0 or 1 sulfur atom, said aromatic or heteroaromatic ring having ortho-substituents selected from —CN, —NO₂, —CF₃, or —OR²;
Ar² is selected from phenyl or pyridyl, and
stereoisomers, enantiomers, in vivo-hydrolysable precursors and pharmaceutically-acceptable salts thereof.

6. A compound according to claim 1, wherein:
D is O; or an enantiomer thereof, and pharmaceutically-acceptable salts thereof.

7. A compound according to claim 1, wherein:
Ar¹ is selected from phenyl or thiophenyl and Ar² is selected from phenyl, pyridyl, furanyl or thiophenyl having optional substituents as defined herein.

8. A compound according to claim 1, selected from:
N-(R)-1-Azabicyclo[2.2.2]oct-3-yl-2-methyl-5-phenylbenzamide;
N-(R)-1-Azabicyclo[2.2.2]oct-3-yl-2-methyl-3-phenylbenzamide;
(N-(R)-1-Azabicyclo[2.2.2]oct-3-yl)-3-methyl-5-phenylthiophene-2-carboxylic acid amide;
(N-(R)-1-Azabicyclo[2.2.2]oct-3-yl)-3-methyl-5-(3-pyridyl)thiophene-2-carboxylic acid amide;
N-(R)-1-Azabicyclo[2.2.2]oct-3-yl-2-carboxy-5-phenylbenzamide;
N-(R)-1-Azabicyclo[2.2.2]oct-3-yl-2-carboxy-3-phenylbenzamide;
(N-(R)-1-Azabicyclo[2.2.2]oct-3-yl)-3-carboxy-5-phenylthiophene-2-carboxylic acid amide;
(N-(R)-1-Azabicyclo[2.2.2]oct-3-yl)-3-carboxy-5-(3-pyridyl)thiophene-2-carboxylic acid amide;
N-(R)-1-Azabicyclo[2.2.2]oct-3-yl-2-cyano-5-phenylbenzamide;
N-(R)-1-Azabicyclo[2.2.2]oct-3-yl-2-cyano-3-phenylbenzamide;
(N-(R)-1-Azabicyclo[2.2.2]oct-3-yl)-3-cyano-5-phenylthiophene-2-carboxylic acid amide;
(N-(R)-1-Azabicyclo[2.2.2]oct-3-yl)-3-cyano-5-(3-pyridyl)thiophene-2-carboxylic acid amide;
N-(R)-1-Azabicyclo[2.2.2]oct-3-yl-2-amino-5-phenylbenzamide;
N-(R)-1-Azabicyclo[2.2.2]oct-3-yl-2-amino-3-phenylbenzamide;
(N-(R)-1-Azabicyclo[2.2.2]oct-3-yl)-3-amino-5-phenylthiophene-2-carboxylic acid amide, or
(N-(R)-1-Azabicyclo[2.2.2]oct-3-yl)-3-amino-5-(3-pyridyl)thiophene-2-carboxylic acid amide.
or pharmaceutically-acceptable salts thereof.

9. A method of treatment or prophylaxis of a disease or condition in which activation of the α7 nicotinic receptor is beneficial which method comprises administering a therapeutically-effective amount of a compound according to claim 1 to a subject suffering from said disease or condition.

10. The method of claim 9, wherein said disease or condition is anxiety, schizophrenia, mania or manic depression.

11. A method of treatment or prophylaxis of neurological disorders, psychotic disorders or intellectual impairment disorders, which comprises administering a therapeutically effective amount of a compound according to claim 1.

12. The method of claim 11, wherein said disorder is Alzheimer's disease, learning deficit, cognition deficit, attention deficit, memory loss, Attention Deficit Hyperactivity Disorder, Parkinson's disease, Huntington's disease, Tourette's syndrome, neurodegenerative disorders in which there is loss of cholinergic synapses, jetlag, nicotine addiction, craving, pain, or ulcerative colitis.

13. A method for inducing the cessation of smoking comprising administering an effective amount of a compound according to claim 1.

14. A pharmaceutical composition comprising a compound according to claim 1 and a pharmaceutically-acceptable diluent, lubricant or carrier.

15. A method of treatment or prophylaxis of a disease or condition in which activation of the α7 nicotinic receptor is beneficial which method comprises administering a therapeutically-effective amount of a pharmaceutical composition according to claim 14 to a subject suffering from said disease or condition.

16. The method of claim 15, wherein said disease or condition is anxiety, schizophrenia, mania or manic depression.

17. A method of treatment or prophylaxis of neurological disorders, psychotic disorders or intellectual impairment disorders, which comprises administering a therapeutically effective amount of a pharmaceutical composition according to claim 14.

18. The method of claim 15, wherein said disorder is Alzheimer's disease, learning deficit, cognition deficit, attention deficit, memory loss, Attention Deficit Hyperactivity Disorder, Parkinson's disease, Huntington's disease, Tourette's syndrome, neurodegenerative disorders in which there is loss of cholinergic synapses, jetlag, nicotine addiction, craving, pain, and for ulcerative colitis.

19. A method for inducing the cessation of smoking comprising administering an effective amount of a pharmaceutical composition according to claim 14.

20. The use of a compound according to claim 1, an enantiomer thereof or a pharmaceutically-acceptable salt thereof, in the manufacture of a medicament for the treatment or prophylaxis of human diseases or conditions in which activation of the α7 nicotinic receptor is beneficial selected from neurological disorders, psychotic disorders, intellectual impairment disorders, Alzheimer's disease, learning deficit, cognition deficit, attention deficit, memory loss, Attention Deficit Hyperactivity Disorder, anxiety, schizophrenia, mania or manic depression, Parkinson's disease, Huntington's disease, Tourette's syndrome, or neurodegenerative disorders in which there is loss of cholinergic synapses.
